(19) United States
(12) Patent Application Publication
(10) Pub. No.: US 2019/0298900 A1
(43) Pub. Date: Oct. 3, 2019

(54) TRANSFUSION SYSTEM AND METHOD

(71) Applicant: Michael Stout, Apex, NC (US)

(72) Inventor: Michael Stout, Apex, NC (US)

(21) Appl. No.: 15/938,768

(22) Filed: Mar. 28, 2018

(51) Int. Cl. A61M 1/02

(52) U.S. Cl. CPC A61M 1/024; A61M 1/0259; A61M 2202/0413; A61M 1/0281

(57) ABSTRACT

A transfusion system is disclosed for use in field, especially emergency situations. It includes two transfusion needles with a tube creating fluid communication between them. A blood counter that alarms or system that turns off the transfusion is included to prevent too much blood being transfused.

### COPYRIGHT NOTICE

A portion of the disclosure of this patent contains material that is subject to copyright protection. The copyright owner has no objection to the reproduction by anyone of the patent document or the patent disclosure as it appears in the Patent and Trademark Office patent files or records, but otherwise reserves all copyright rights whatsoever.

### BACKGROUND OF THE INVENTION

### Field of the Invention

The present invention relates blood transfusions and devices for transfusions. In particular, it relates to a transfusion system that decreases the time it takes for a transfusion, especially in field (such as during military operations in the field) transfusions.

### Description of Related Art

A transfusion is a traumatic event where blood is removed from one individual, animal, or human, and transfused to another in need. Since transfusion is a traumatic event, the longer it takes to complete the transfusion, the more traumatic the process is.

The current way blood is transfused, especially in the field, is first to remove blood from a donor into a “blood bag”. Typical blood bags hold about 450 to 500 mls of blood. After removal of the blood, the blood bag is connected to a “y” tubing along with a bag of saline or other appropriate solution. Both lines then are opened so that blood and saline travels to the recipient's veins. The process is completed once the blood bag is empty and the liquid solution has flushed the line. Typically this process takes about 30 to 40 minutes.

The more complex the transfusion process, the more complications can arise. The slower the process, likewise, is fraught with complications. With the bag, if more than 500 ml is necessary, the more complicated it gets with multiple bags being necessary. Accordingly, it would be of great use if the process could be made faster with less complexity. It would also be useful if an in the field transfusion could be done faster.

### BRIEF SUMMARY OF THE INVENTION

The present invention involves a system and method that reduces the time to do a transfusion between two individuals to the 5 to 10 minute time frame from 20 minutes or so. This is especially useful in the field such as in a military situation, a wilderness situation, a “buddy” transfusion, an emergency, and the like where there is no medical facility close by though the device can be used for any transfusion. A direct transfusion method with a blood counter which alarms or turns off when the required amount of blood is transfused is disclosed.

Accordingly, in one embodiment, there is a system for transfusing blood from a donor to a recipient comprising:
a) a first transfusion needle for insertion into a vein of the donor;
b) a second transfusion needle for insertion into a vein of the recipient;
c) one or more pieces of tubing connected to the first and second transfusion needles in fluid communication such that blood can flow from the donor to the recipient directly; and
d) a blood counter, which measures the amount of blood transfused, can be set for the amount of blood to be transfused and can do at least one of the following: stops when the counter reaches the set amount of blood consisting of: sounding an alarm that the transfer amount has been reached and blocking further transfer of blood from the donor to the recipient.

In yet another embodiment, there is a method of transferring blood from a donor to a recipient comprising:
a) inserting a first needle which first needle is connected to a first end of a tube into a vein of a donor;
b) inserting a second needle which second needle is connected to a second end of the tube into the vein of a recipient, wherein insertion of the second needle initiates the transfusion of blood between the donor and the recipient;
c) positioning a blood counter in a position to measure the amount of blood transfused from the donor to the recipient which can do at least one of the following: stops when the counter reaches the set amount of blood consisting of: sounding an alarm that the transfer amount has been reached and blocking further transfer of blood from the donor to the recipient; and
d) removing the first and second needles from the donor and recipient respectively when the transfusion is completed.

### BRIEF DESCRIPTION OF THE DRAWINGS

FIG. 1 is a drawing of the system device of the invention.

FIG. 2 is a flow chart of the method of the present invention.

FIG. 3 is a perspective view of a donor and recipient in a transfusion process.

### DETAILED DESCRIPTION OF THE INVENTION

While this invention is susceptible to embodiment in many different forms, there is shown in the drawings, and will herein be described in detail, specific embodiments with the understanding that the present disclosure of such embodiments is to be considered as an example of the principles and not intended to limit the invention to the specific embodiments shown and described. In the description below, like reference numerals are used to describe the same, similar, or corresponding parts in the several views of the drawings. This detailed description defines the meaning of the terms used herein and specifically describes embodiments in order for those skilled in the art to practice the invention.

### Definitions

The terms “about” and “essentially” mean ±10 percent.

The terms “a” or “an”, as used herein, are defined as one or as more than one. The term “plurality”, as used herein, is defined as two or as more than two. The term “another”, as used herein, is defined as at least a second or more. The terms “including” and/or “having”, as used herein, are defined as comprising (i.e., open language). The term “coupled”, as used herein, is defined as connected, although not necessarily directly, and not necessarily mechanically.

The term “comprising” is not intended to limit inventions to only claiming the present invention with such comprising language. Any invention using the term comprising could be separated into one or more claims using “consisting” or “consisting of” claim language and is so intended.

Reference throughout this document to “one embodiment”, “certain embodiments”, “an embodiment”, or similar terms means that a particular feature, structure, or characteristic described in connection with the embodiment is included in at least one embodiment of the present invention. Thus, the appearances of such phrases or in various places throughout this specification are not necessarily all referring to the same embodiment. Furthermore, the particular features, structures, or characteristics may be combined in any suitable manner in one or more embodiments without limitation.

The term “or” as used herein is to be interpreted as an inclusive or meaning any one or any combination. Therefore, “A, B, or C” means any of the following: “A; B; C; A and B; A and C; B and C; A, B, and C”. An exception to this definition will occur only when a combination of elements, functions, steps, or acts are in some way inherently mutually exclusive.

The drawings featured in the figures are for the purpose of illustrating certain convenient embodiments of the present invention, and are not to be considered as limitation thereto. Term “means” preceding a present participle of an operation indicates a desired function for which there is one or more embodiments, i.e., one or more methods, devices, or apparatuses for achieving the desired function and that one skilled in the art could select from these or their equivalent in view of the disclosure herein and use of the term “means” is not intended to be limiting.

As used herein, the term “donor” refers to a human or animal donating blood.

As used herein, the term “recipient” refers to a corresponding human or animal receiving blood.

As used herein, the term “transfusing blood” refers to the process of taking blood from a donor person or animal and transferring it to a respective person or animal. Transfusions are used for various medical conditions to replace lost components of the blood. In the present invention, it refers only to whole blood transfusions since any intermediary steps have been eliminated. A filter may also be added to the system such that blood flows through the filter to remove any clots or other unwanted material. Typical filters are 170 to 260 microns and are well known in the art and are placed in line of the flow of blood. Transfusion is initiated when needles are in both donor and recipient, however, in some embodiments, hose clamps, or other devices that block blood flow are used on the tubing to prevent the transfusion from starting before desired. Transfusion would then begin when the clamp or block is removed.

As used herein, the term “transfusion needle” refers to a sterile surgical needle standard for blood transfusions. Such needles are well known in the art. Needle size depends on the size and integrity of a donor's or recipient's vein. An 18-gauge needle is standard, but a needle or catheter as small as 23-gauge can be used for transfusion if necessary. The smaller the gauge, the slower the flow rate and the higher the risk of clotting. Care must be taken to avoid excessive pressure resulting in hemolysis when very narrow needles are used.

As used herein, the term “tubing” refers to medical tubing of the type generally accepted for use in doing blood transfusions. Such tubing is readily available. In the practice of the invention, a first transfusion needle attaches to one end of the tubing and a second transfusion needle is attached to the second end with the first needle inserted in the vein of the donor and the second needle inserted in the vein of the recipient. In one embodiment, there is a single piece of tubing. In other embodiments, there can be multiple pieces of tubing connected together in fluid communication. In one embodiment, the blood counter is spliced into the tubing such that blood flows through the counter for measurement. The material and length of the tubing can be determined by one skilled in the art in view of this disclosure.

As used herein, the term “blood counter” refers to a device which can determine how much blood is flowing from the donor to the recipient. It can do that by direct measure, mechanical or digital, of the blood flowing through the blood counter. In other embodiments, the blood counter is placed next to the tubing and can measure blood flow, such as by ultra sound. Since one needs to monitor the amount transfused, there is an input device on the blood counter to let the user set the amount to be transfused and to let the user know when that amount is reached. In one embodiment wherein the blood flows through the counter, once the desired amount of blood is transfused, the counter blocks the flow of blood. In some embodiments, only an alarm sounds and the user must then clamp or otherwise stop the flow of blood. Where the power is needed for the blood counter or the system, AC or DC power is included. In another embodiment, the blood counter detects back flow and either notifies users or blocks the blood flow or both.

In the process of the invention, the first transition needle is inserted into the vein of the donor. The second transfusion needle is inserted into the vein of the recipient. Blood flow is prevented by clamping the tubing connecting the two needles. The blood counter is positioned next to or flow through such that the quantity of transfused blood is measured. In other embodiments, the blood counter can be set to have an alarm when a designated amount of blood is transfused. In other embodiments, the blood counter can shut off the blood flow automatically. Blood flow commences and once completed, the needles are removed from donor and recipient.

### Drawings

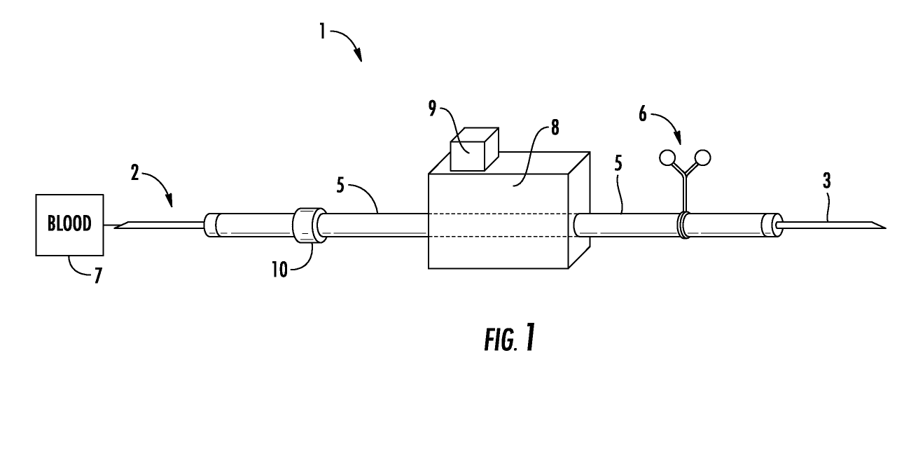

Now referring to the drawings, FIG. 1 is a perspective view of the device of the present invention. Device 1 comprises a donor transfusion needle 2 and a recipient transfusion needle 3. The two needles 2 and 3 and in fluid communication via tube 5 (which can be multiple pieces though only one is shown). Clamp 6 is shown which prevents the start or ends flow, where necessary. Blood 7 enters the donor needle 2 and flows through the tubing to recipient needle 3. Along the tubing, blood counter 8 is shown. The tubing can run through the counter and the counter can shut off the flow. In other embodiments, the counter is placed next to the tube and senses the blood flow. A controller 9 is shown which can have one or more functions, including setting an amount of blood to transfer, sounding an alarm, cutting off blood flow, detecting back flow, and the like. In this view, there is blood filter 10 for filtering blood before reaching recipient.

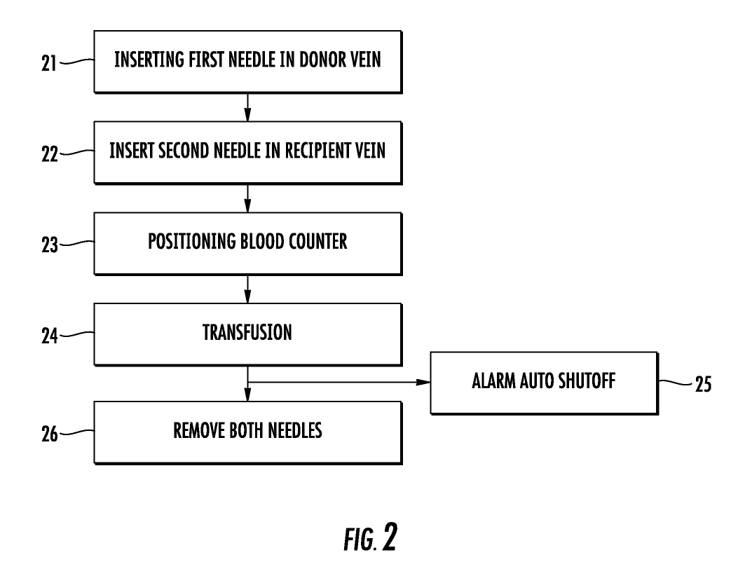

FIG. 2 is a flow chart of the method of using the device of the present invention. The device process starts with inserting a needle in the donor vein 21 making sure the tubing is clamped shut. The recipient transfusion needle is then inserted in the recipient vein 22. The blood counter is positioned 23 on or next to the tubing, though it could be prepositioned or attached to the tubing or the like, as desired. The clamp or other device that regulates flow is then opened/removed and transfusion 24 of blood begins. Once the appropriate amount of blood has been transfused, the blood counter sounds an alarm or engages an auto shutoff 25, stopping the transfusion. The two needles are then removed 26, ending the transfusion process.

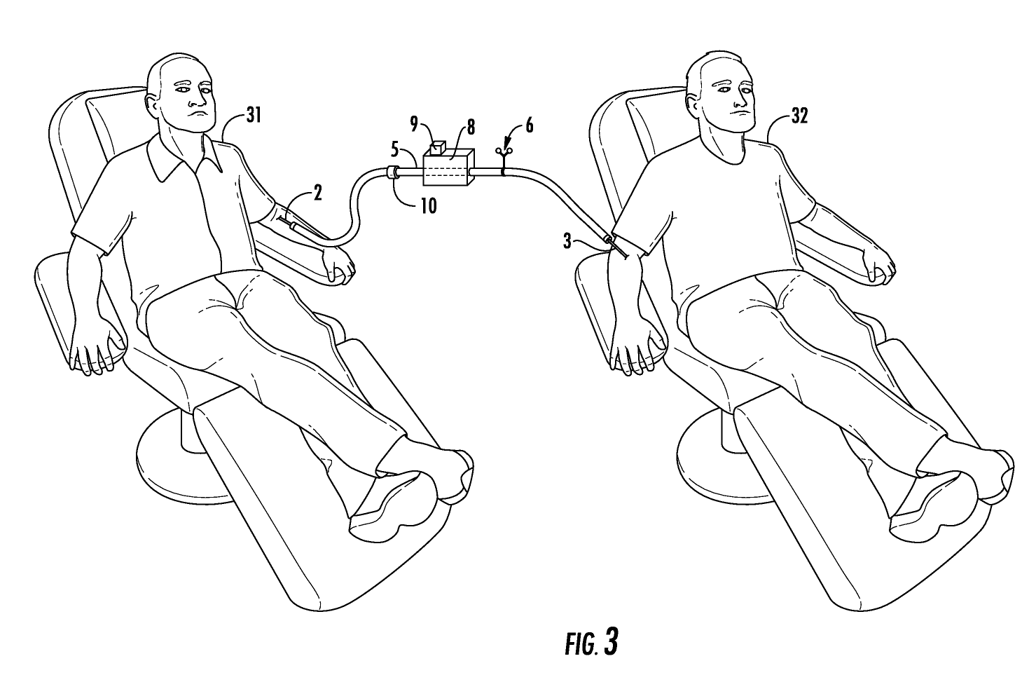

FIG. 3 is a perspective view of a donor transfusing blood to a recipient using the device of the present invention. While humans are shown, animals are also intended. Parts are numbered as in FIG. 1. Donor 31 has the needle 2 inserted in a vein to remove blood 7. Tubing 5 is clamped 6 to prevent blood from flowing. The recipient needle 3 is inserted in recipient 32. The blood counter 8 is programmed for how much blood is to be transfused using controller 9 and then the clamp is removed starting the process. When the counter 8 senses the transfusion amount has been reached, controller 9 either sounds an alarm or, in one embodiment, shuts off the flow of blood. In one embodiment, instead of a clamp, the blood controller could have an open and closed position which can prevent blood flowing through the tube.

Those skilled in the art to which the present invention pertains may make modifications resulting in other embodiments employing principles of the present invention without departing from its spirit or characteristics, particularly upon considering the foregoing teachings. Accordingly, the described embodiments are to be considered in all respects only as illustrative, and not restrictive, and the scope of the present invention is, therefore, indicated by the appended claims rather than by the foregoing description or drawings. Consequently, while the present invention has been described with reference to particular embodiments, modifications of structure, sequence, materials, and the like apparent to those skilled in the art still fall within the scope of the invention as claimed by the applicant.

What is claimed is:

1. A system for transfusing blood from a donor to a recipient comprising:
a) a first transfusion needle for insertion into a vein of the donor;
b) a second transfusion needle for insertion into a vein of the recipient;
c) one or more pieces of tubing connected to the first and second transfusion needles in fluid communication such that blood can flow from the donor to the recipient directly; and
d) a blood counter, which measures the amount of blood transfused, can be set for the amount of blood to be transfused and can do at least one of the following: stops when the counter reaches the set amount of blood consisting of: sounding an alarm that the transfer amount has been reached and blocking further transfer of blood from the donor to the recipient.

2. The system according to claim 1 wherein the blood counter measures by flowing the blood transfer through the blood counter.

3. The system according to claim 1 wherein the blood counter measures by flowing blood next to the blood counter.

4. The system according to claim 1 wherein the blood counter can detect back flow during the transfusion.

5. The system according to claim 1 wherein there are clamps or other devices to block blood flow.

6. A method of transferring blood from a donor to a recipient comprising:
a) inserting a first transfusion needle which first needle is connected to a first end of a tube into a vein of a donor;
b) inserting a second transfusion needle which second needle is connected to a second end of the tube into the vein of a recipient, wherein insertion of the second needle initiates the transfusion of blood between the donor and the recipient;
c) positioning a blood counter in a position to measure the amount of blood transfused from the donor to the recipient which can do at least one of the following: stops when the counter reaches the set amount of blood consisting of: sounding an alarm that the transfer amount has been reached and blocking further transfer of blood from the donor to the recipient; and
d) removing the first and second needles from the donor and recipient respectively when the transfusion is completed.

7. The method according to claim 6 which further comprises clamping or blocking the tubing to prevent blood flow.
